(19) United States
(12) Patent Application Publication
(10) Pub. No.: US 2024/0374249 A1
(43) Pub. Date: Nov. 14, 2024

(54) METHOD OF GRAFT PREPARATION

(71) Applicants: Smith & Nephew, Inc., Memphis, TN (US); Smith & Nephew Orthopaedics AG, Zug (CH); Smith & Nephew Asia Pacific Pte. Limited

(72) Inventors: Elizabeth A. Capogna, Wrentham, MA (US); Christopher D. MacCready, Medfield, MA (US); Gregory Gorham, Billerica, MA (US)

(21) Appl. No.: 18/637,796

(22) Filed: Apr. 17, 2024

Related U.S. Application Data
(60) Provisional application No. 63/460,480, filed on Apr. 19, 2023.

(51) Int. Cl. A61B 17/04

(52) U.S. Cl. CPC A61B 17/0401; A61B 2017/0446

(57) ABSTRACT

Methods of coupling a suspensory fixation construct to a graft, to avoid graft folding while being drawn into a bone tunnel. The suspensory fixation construct may include an adjustable loop construct and a passing construct linked thereto. The method may include forming a running whipstitch along the graft and over a length of the adjustable loop construct such that the running whipstitch holds the adjustable loop construct length along and against the graft surface. The method may include coupling a portion of the passing construct to the adjustable loop construct after the suspensory fixation construct has been attached to the graft.

### CROSS-REFERENCE TO RELATED APPLICATIONS

This application is a U.S. application which claims the benefit to U.S. Provisional App. No. 63/460,480 filed Apr. 19, 2023, titled “METHOD OF COUPLING AN ADJUSTABLE SUSPENSION CONSTRUCT TO A GRAFT” herein incorporated by reference in its entirety.

### FIELD

The present disclosure relates to methods and devices for joint repair, including graft fixation in a surgical repair.

### BACKGROUND

Native soft tissue (such as a ligament of a joint) that is damaged may generally be replaced or repaired arthroscopically. For some joint repairs, a tissue fixation system that includes a suture construct may be coupled to a graft and drawn through a bone tunnel. The suture construct may form an adjustable loop construct that may be adjusted to reduce a loop size and position the graft in a target location along the bone tunnel. The adjustably loop construct may be coupled to a tissue anchor, such as a cortical button. Example adjustable loop constructs with cortical buttons are disclosed in at least PCT Application No. PCT/US2022/026320, filed Apr. 26, 2022, commonly owned and titled “DEVICES AND METHODS FOR JOINT REPAIR”, herein incorporated by reference in its entirety. As smaller tunnels are preferable to remove less bone, careful preparation of the graft and the graft coupling to the suture construct is required. Without this preparation, the graft may be damaged while being pulled into these smaller tunnels. Related art techniques of graft coupling may allow the inadvertently graft to fold over itself and hinder passage of the graft along these smaller tunnels. Overcoming this may damage the graft. Should the suture construct be adjustable, a folded graft may hinder any sliding of the suture and therefore any reduction of the suture construct. Therefore, there is a need for an improved coupling technique of the tissue fixation construct to the graft that addresses the related art shortcomings.

### SUMMARY

Described herein are improved techniques for coupling a suture of a suspension fixation construct to a graft end. These improved techniques may prevent the graft end from folding over as it is drawn along a bone tunnel.

An example method of coupling a suspensory fixation system to a graft is disclosed, the suspensory fixation system including an adjustable loop construct with a passing loop construct linked thereto. The method may include using the passing construct to stitch the adjustable loop construct through and around the graft at a first location along the graft. The passing construct may then be stitched through the graft adjacent the first location to secure the adjustable loop construct to the graft. With the adjustable loop construct secured, a length of the adjustable loop may be placed along an outer surface of the graft and the passing loop construct may be stitched around and through the graft at a second location along the graft, the second location disposed between the first location and a free end of the graft. Stitching the passing loop construct at the second location may also simultaneously wrap a strand of the passing loop construct around and over the length of the adjustable loop construct so that it holds the length of the adjustable loop firmly against the graft adjacent the second location.

Furthermore, in some example methods, the first location may be spaced away from the graft free end. The method may also include stitching the passing loop construct around and through the graft at a third location along the graft, the third location disposed between the second location and the free end of the graft, and this may wrap the passing loop construct a second time around and over the length of the adjustable loop construct to hold the length of the adjustable loop firmly against the graft adjacent the third location. The example method may include coupling an end of the passing loop construct to the adjustable loop construct after stitching the passing construct around and through the graft at the second location. The adjustable loop construct may be drawn along a bone tunnel after coupling the passing construct to the adjustable loop construct, to drawing the graft free end along the bone tunnel. The adjustable loop construct may include a static leg and coupling the passing loop construct to the adjustable loop construct may couple a flexible strand of the passing loop construct to the static leg. The static leg may be marked. In other example methods, the static leg may not be marked but determined by reducing the adjustable loop construct slightly, to observe which strand leg is static before coupling the flexible strand of the passing construct to the static leg. Coupling the flexible strand of the passing construct to the static leg may include stitching the flexible strand through the static leg. The method may include drawing the adjustable loop construct along a bone tunnel, and thereby drawing the graft free end along the bone tunnel. The length of the adjustable loop construct may include multiple loop lengths of the adjustable loop construct and wherein stitching the passing construct at the second location may inserts the passing construct through the graft at the second location and split or separate some of the multiple loop lengths so that the multiple loop lengths extend either side of the second location.

Another example method of coupling a graft suspension system to a graft is disclosed. This method may include obtaining the graft and the graft suspension system, the graft suspension system including a tissue anchor with a plurality of openings therethrough, an adjustable loop construct formed with a flexible strand, a first end of the adjustable loop construct coupled to the tissue anchor via the plurality of openings, and the flexible strand including at least one locking passage. The graft suspension construct may also include a passing construct including a flexible strand and a needle, the flexible strand separately formed from the adjustable loop construct flexible strand and coupled to a second end of the adjustable loop construct defining a first link, the second end an opposing end to the first end. The method may include coupling the adjustable loop construct to the graft at a first location along the graft and coupling the passing construct to the graft at a second location along the graft. The method may also include linking the passing construct flexible strand to the adjustable loop construct after coupling the adjustable loop construct to the graft at the first location and coupling the passing construct to the graft at the second location, such that the passing construct and the adjustable loop construct are linked by the first link and a second link, at separate locations from each other. The method may also include drawing the graft suspension system and the graft through a bone tunnel.

Furthermore, in some example methods, coupling the passing construct may include forming at least two whipstitches along the graft free end with the passing construct. Linking the passing construct flexible strand to the adjustable loop construct may comprise coupling the passing construct flexible strand to a static leg of the adjustable loop construct. Linking the passing construct flexible strand to the adjustable loop construct may comprise at least one of passing or splicing the passing construct flexible strand to the adjustable loop construct. Linking may include passing a first end of the passing construct flexible strand through the adjustable loop construct flexible strand at a first location and also passing a second end of the passing construct flexible strand through the flexible strand of the adjustable loop construct at a second, different location. The adjustable loop construct may include a static leg, and linking may include passing an end of the passing construct flexible strand through the static leg. The adjustable loop construct may be reduced slightly, sufficiently to identify the static leg before linking. The static leg may be obtained marked, for identifying the static leg. While coupling the passing construct to the graft at a second location along the graft; the passing construct flexible strand may be passed over a length of the adjustable loop construct to hold this length against the graft external surface.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The disclosure will be more fully understood by reference to the detailed description, in conjunction with the following figures, wherein:

FIG. 1 illustrates an example graft suspension construct including an adjustable loop construct with a passing loop construct coupled thereto;

FIGS. 2A-2C illustrate forming the adjustable loop construct shown in FIG. 1;

FIGS. 3A-3J illustrate some of the steps for coupling a graft suspension construct to a graft in accordance with this disclosure;

FIG. 4A illustrates a top view of the step shown in FIG. 3E;

FIG. 4B illustrates a top view of the arrangement after the step shown in FIG. 3E;

FIGS. 5A-5C illustrate a top view of a first example method of further coupling a graft suspending construct coupled to the graft as shown in FIG. 4B to reduce folding of the graft in accordance with this disclosure;

FIG. 5D illustrates drawing the graft through a bone tunnel, the graft coupled to the graft suspension construct as disclosed in FIG. 50;

FIGS. 6A-6D illustrates some example routings for coupling the passing sloop strands to the adjustable construct static strand, in accordance with this disclosure;

FIGS. 7A-7B illustrate a side view of another example method of coupling a graft suspending construct, to avoid graft folding in accordance with this disclosure; and

FIG. 7C illustrates a top view of FIG. 7B.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open ended and include the listed parts and can include additional parts that are not listed. “And/or” is open-ended and includes one or more of the listed parts and combinations of the listed parts. Use of the terms “upper,” “lower,” “upwards,” and the like is intended only to help in the clear description of the present disclosure and are not intended to limit the structure, positioning and/or operation of the disclosure in any manner.

Some of the constructs disclosed herein incorporate at least one “locking passage”. Locking passages may otherwise by referred to in the art as splices, eyesplices, cradles, suture locking regions, cinches, finger cinches, finger traps, longitudinal passages, or dilated regions. They are defined by a length of a braided flexible strand with a hollow core that may receive an elongate flexible strand therethrough. The elongate flexible strand may be a different portion of the flexible strand, or another separate flexible strand and may extend along a path that extends from outside the braided flexible strand (and outside the locking passage) then between the braids to enter the hollow core (lumen) and then exit through the braided wall a distance along the braided flexible strand later. The elongate flexible strands may extend along and through the hollow core at multiple spaced apart locations, thereby defining multiple locking passages. Multiple lengths of an elongate strand or strands may extend along and through the hollow core at the same location. The braided flexible strand may be dilated first to form a dilated or laterally extended length before receiving the elongate flexible strand therein. The locking passage is configured such that tension on the braided hollow flexible strand contracts the inner core radius thereof and thereby locks or cinches around the elongate flexible strand extending therein, locking the elongate flexible strand in place (preventing it from sliding along the hollow core). This defines a “locking passage”. The flexible strand may be a suture, suture tape, ribbon, or flexible tubular cable.

A “static leg” or “stationary leg” is defined as a portion of the flexible strand that is part of the adjustable loop construct that remains stationary or static relative to a graft during loop reduction. It may also remain static relative to a locking passage (as defined herein). For adjustable loop constructs, loop reduction is achieved by tensioning an end or limb of the adjustable loop construct, and during reduction some non-static portions of the adjustable loop construct may slide relative to the graft, while static portions may move also during reduction, they do not move relative to the graft, as coupled to the adjustable loop construct. Oftentimes, the static strand portion is continuously braided with and extends directly from the locking passage.

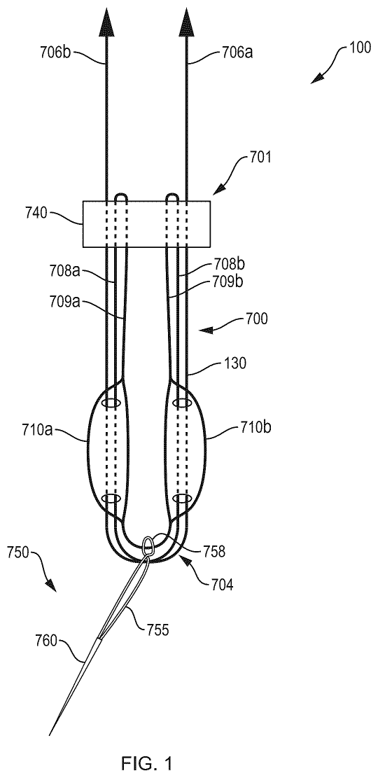

As discussed herein fixation systems or graft suspension systems with cortical buttons may operate to couple to and suspend a graft within and along a bone tunnel of an articulating joint. An example graft suspension system 100 is illustrated in FIG. 1. System 100 may include an adjustable loop construct 700 and a second or passing loop construct 750. Adjustable loop construct 700 may include two locking passages 710a, 710b, and may be assembled, partially assembled directly to a tissue anchor such as a cortical button 740. Adjustable loop construct 700 may be provided separately from a tissue anchor. Adjustable loop construct 700 may be formed from a single flexible strand 130. Adjustable loop construct 700 may be formed by a flexible strand 130 that is a braided suture, braided to be hollow (in at least some portions) defining an elongate passage or lumen therealong. Adjustable loop construct 700 may be preassembled to at least one side of button 740. Adjustable loop construct may include a saddle end 704 and two free ends 706a and 706b. Tension on free ends 706a, 706b may adjust or reduce the loop size. Stated another way, tension on the free ends 706a, 706b may draw the saddle end 704 towards the button 740. Upon a graft being coupled to the saddle end 704, tension on free ends 706a, 706b draws the graft towards the button 740.

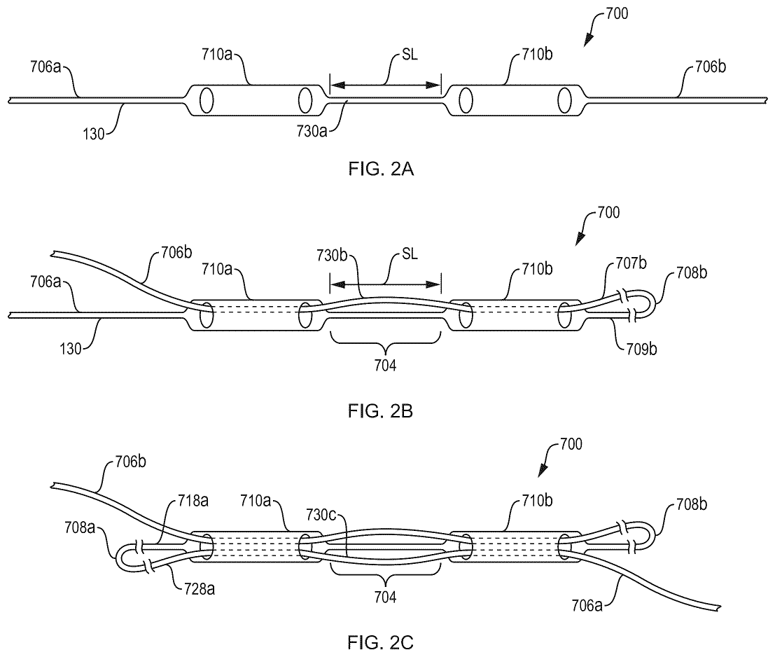

FIG. 2A-2C demonstrate the steps to forming the example adjustable loop construct 700. Beginning with FIG. 2A, a length of a flexible strand 130 is shown. Locations of locking passages 710a, 710b are shown as enlarged or dilated portions for clarity of discussion. However, as provided, these locations may be similar in diameter and shape to the remaining length of the strand 130 and the act of spicing the strand 130 and extending the strand 130 through itself may dilate that portion of the strand 130. A snare loop (not shown) may extend along passages 710a, 710b. A dilating means (not shown) may be first extended through passage (710a, 710b) locations.

Turning now to FIG. 2B, end 706b may be extend into and along the strand 130 at a first end of locking passage 710b, extend along the strand lumen and then exit the passage 710b, remaining external to the strand lumen for a length (approximately SL). End 70bB may then extend back into, along and out of lumen of strand 130 at locking passage location 710a. This forms a first eyesplice loop 708b and end 706b. The length SL between the two locking passages 710a, 710b may be selected, depending on the procedure or application. For example, if the saddle end 704 is configured to couple to a graft, the saddle length SL may be long enough to wrap around the graft width, with the locking passages 710a, 710b disposed along a side of the graft. While forming this first eyesplice loop 708b, the loop may be threaded through apertures of a button. This may assemble the construct 700 with button 740. Eyesplice loop 708b may define a static leg 709b as defined herein. Static leg 709b may be continuously braided directly from locking passage portion 710b. Eyesplice loop 708b may define a sliding leg 707b that extends into the hollow passage of the passage portion 710b. These legs 709b, 707b are continuous with each other. In some embodiments 709b may be provided marked or indicated, as described hereinafter.

FIG. 2C shows formation of a second eyesplice loop 708a. Similar to first eyesplice loop 708a, limb end 706a may extend into and along the strand 130 at locking passage 710a first, forming the second eyesplice loop 708a. Limb end 706a may than extend for a length along saddle end 704 before extending into and along locking passage 710b. Second eyesplice loop 708a may also thread through apertures of a button, such as for example button 740. This assembles both loops 708a, 708b to a button. Eyesplice loop 708a may define a static leg 709a that may extend directly from locking passage portion 710a. Eyesplice loop 708a may define a sliding leg 707a that extends into the hollow passage of the locking passage 710a. These legs 707a, 709a may be continuous with each other. In some embodiments 709a may be provided marked or indicated, as described hereinafter.

In this construct 700, each locking passage 710a, 710b includes two lengths of strand 130 extending therethrough. The two lengths of strand 130 cross over each other to exit from opposite ends of each locking passage 710a, 701b. In other embodiments, each limb may only extend through one locking passage 710a, 710b. For example, limb 706b may extend through passage 710b only to form eyesplice loop 708a. The distance between to the two discrete locking passages 710a, 710b may be sufficient to extend through a thickness of a graft. Saddle end 704 may include three strand lengths of strand 130, two of which are slideable and one single strand which is static or fixed (non slideable) and extends directly from, and is continuously braided with both locking passages 710a, 710b.

Construct 700 is an example adjustable loop construct. Other adjustable loop constructs known in the art are disclosed in at least commonly owned PCT Application No. PCT/US2022/026320, filed Apr. 26, 2022, and titled “DEVICES AND METHODS FOR JOINT REPAIR”, commonly owned PCT patent application number PCT/US20/038401 filed Jun. 18, 2020, or commonly owned U.S. Pat. No. 10,383,617, filed Feb. 4, 2017, also incorporated by reference in their entirety.

Returning to FIG. 1, system 100 may also include a passing loop construct 750, which may be coupled to the saddle end 704. The passing loop construct 750 is configured to draw the adjustable loop construct 700 through a tissue or graft and then be form additional stitched through the graft. The additional stitches may increase a coupling strength overall and also reduce the likelihood of cheese wiring of the graft. Passing construct 750 may include a loop 755 formed of a flexible strand such as a suture, suture tape, wire or cable. Loop 755 may be coupled to a threading member 760 and loop 755 may having a fixed length (non-adjustable). Threading member 760 may be a needle that pierces the tissue or graft. Threading member 760 may be configured to pierce a tissue or graft and draw the loop 755 followed by the saddle end 704 of adjustable loop construct 700 therethrough.

Loop 755 may be formed, starting with a length of suture, with two terminal ends that are swaged or crimped to threading member 760, to form the loop 755. Loop 755 may be formed of a flexible strand that is different from and/or separately formed from flexible strand 130, for example is may not include a hollow core. Passing construct 750 may be coupled to the three strand lengths of the saddle end 704 with a complex loop 758. The complex loop 758 may be configured to sequentially draw the three strand lengths through the tissue. Sequentially drawing the strand lengths of the saddle end 704 may reduce tissue damage or deformation, and reduce forces required to draw the saddle end 704 through the tissue or graft. This passing construct 750 may couple the saddle end 704 to the tissue or graft. The complex loop 758 may be configured to limit sliding of the passing construct 750 along the adjustable loop construct 700. The complex loop may help to control the three length strands and maintains them in close approximation as they slide relative to each other. The complex loop may be a figure-of eight loop.

### Method of Attaching an Adjustable Loop Construct

The disclosure now turns to methods of attaching a suspension construct, such as construct 100. The methods are configured to stitch the suspension construct to a graft and define an effective tensioning location of the suspension construct close to a graft free end 1660, the effective tensioning location being defined by where tension is applied along the graft 1650 as it is drawn through a bone tunnel via pulling on the suspension construct.

An adjustable loop construct may first be obtained. The adjustable loop construct may be similar to construct 700, which may be obtained preassembled with a passing construct 750, similar to construct embodiment 100. In other embodiments the passing construct 750 may be obtained separately and the method may include coupling the two together. Adjustable loop construct 700 may define a first end 701 that couples to a tissue anchor 740, and an opposing end or saddle end 704. Saddle end 704 may couple directly to graft 1650. Passing construct 750 may be coupled to a saddle end 704 with a complex loop 758, as disclosed herein. The method disclosed couples an adjustable loop construct such as construct 700 to a graft 1650, such that the final stitched arrangement through and along the graft 1650 includes both an adjustable loop construct and a passing loop 755 stitched therethrough. Passing loop 755 and adjustable loop construct 700 may both be formed with flexible strands that are formed separately from each other. In the final stitched graft, the passing loop 755 may form a row of running whipstitches that may axially overlap at least some adjustable loop construct stitches. In the final stitched graft, the passing loop 755 may form a row of running whipstitches that cover a length of the adjustable loop construct and hold this length against the graft 1650. In the final stitched graft, the passing loop 755 may be coupled to a strand of the adjustable construct 700 after stitching through the graft, such that while drawing the final stitched graft through the bone tunnel, using the adjustable construct 700, the effective tensioning location is such that the graft free end 1660 is inhibited from folding over on itself.

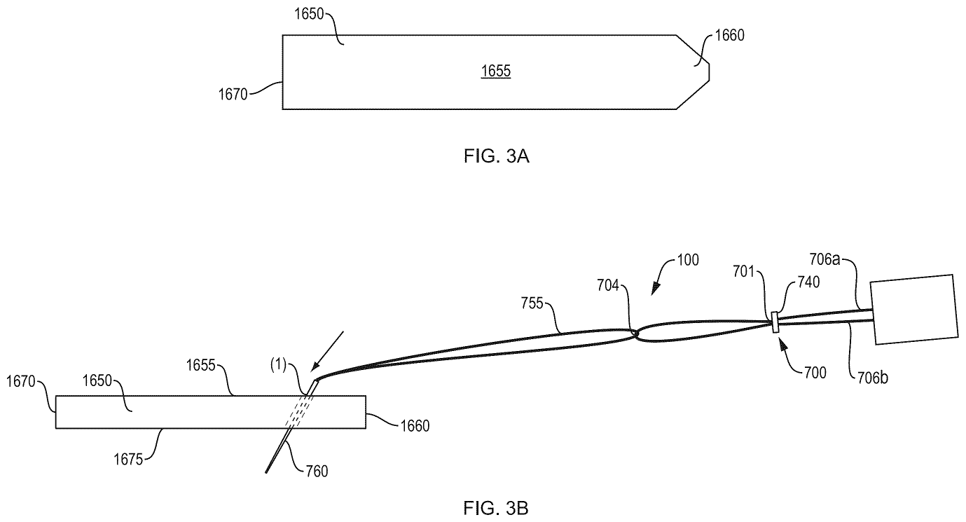

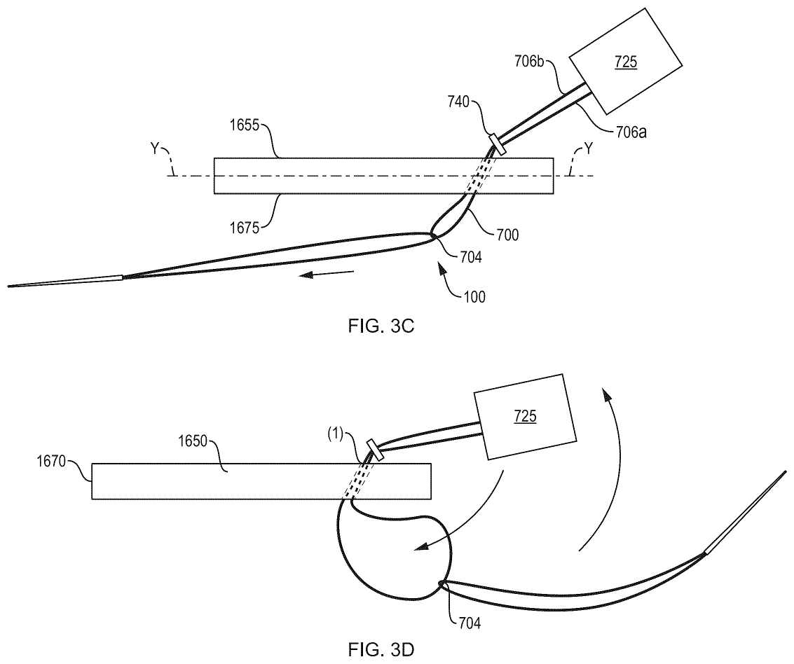

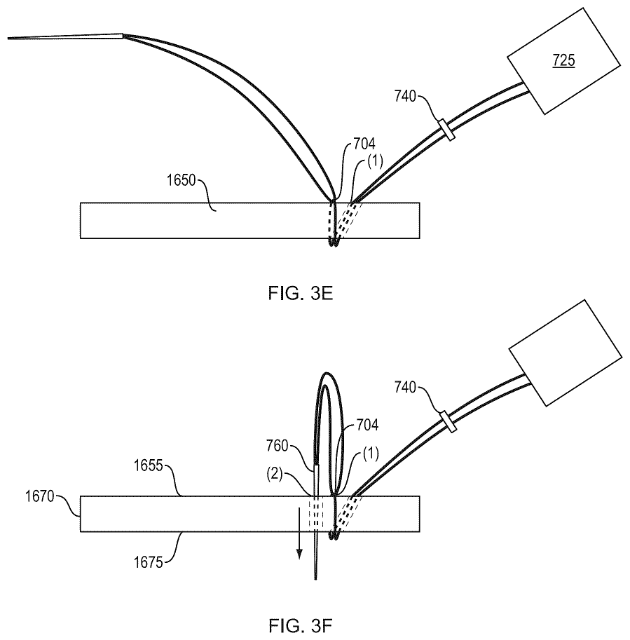

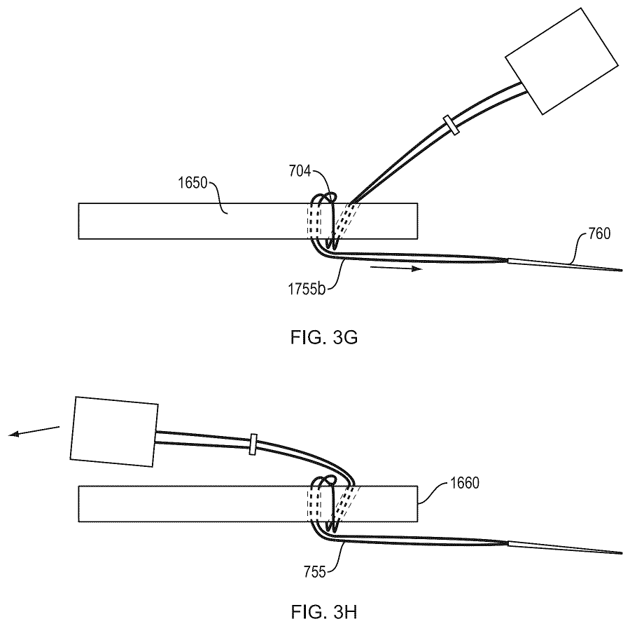

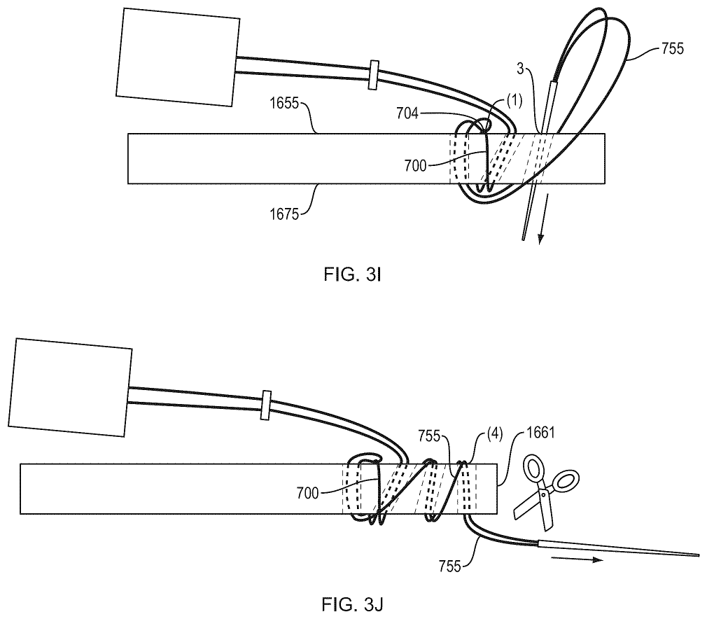

FIGS. 3A-3J all show a simplified form of an adjustable loop construct with some parts of the construct not shown, for simplicity of understanding. For example, not shown are any locking passages (such as but not limited to locking passages 710a, 710b). Adjustable loop construct may include a plurality of adjustable loops (such as but not limited to loops 708a, 708b), also not shown in these figures. These details are omitted from the figures to simplify understanding of the figures and thereby understanding of the method. In addition, the methods disclosed herein may couple other adjustable loop constructs to a graft in this manner, such as those disclosed herein by reference, or others known in the art with differing locking means such as with preformed knots.

Starting with FIG. 3A, the method may include obtaining and/or harvesting a graft 1650. Graft 1650 may be an elongate body, defining a top side 1655, bottom side 1675, and two opposing ends 1660, 1670. One of the two opposing ends (1670) may be clamped to stabilize the graft 1650 during preparation and stitching, and therefore may be defined as a clamped end 1670. The other of the two opposing ends may be the free end 1660. Free end 1660 may enter a bone tunnel first when drawn by the graft suspension construct 100. Free end 1660 may be directly coupled to both the adjustable loop construct 700 and passing construct loop 750. Free end 1660 may be tapered or bulletized using a scalpel or scissors for easier threading through a prepared bone tunnel. Graft 1650 may be a single solid body and may be formed from the patient's Quad Tendon. Graft 1650 may include wispy ends. Graft 1650 may be provided as a plurality of elongate strands, typical when harvesting from the hamstring for example. Graft 1650 may be folded over itself to form a target thickness of graft 1650.

Now turning to FIG. 3B showing a side view of the graft 1650, the method of attaching may include forming and/or obtaining an adjustable loop construct such as construct 700 that may be assembled to a cortical button 740 at one end and assembled to a flexible passing loop 755 of a passing construct 750 at the saddle end 704. The flexible passing loop 755 may be operatively coupled to a passing or threading member 760 such as a needle defining, together, the passing construct 750. Passing member 760 may pierce the graft top surface 1655 and draw the flexible passing loop 755 from the graft top surface 1655 through the thickness of graft 1650 to the lower external surface 1675, at a first location (1). First location (1) may be about 1.5 cm-2 cm from terminal edge 1661 of free end 1660. The first pass may be complete when passing construct 750 is drawn completely through graft 1650, until the entire passing construct 750 is external to the graft 1650 and the adjustable loop construct 700 extends through graft 1650 and from both the top surface 1655 and bottom surface 1675 respectively (FIG. 3C). Adjustable loop construct 700 may include two limbs 706a, 706b wrapped around a carrying card or tool 725 for management thereof. First pass may extend through the graft 1650 at an angle that is neither inclined relative to the longitudinal axis (Y-Y), and therefore neither parallel to nor orthogonal to longitudinal axis (Y-Y) of the graft 1650. Angle may be between 30-60 degrees relative to the longitudinal axis. First pass may extend along and intersect the longitudinal axis. First pass, and all subsequent passes may extend through a midline of the graft 1650, as best possible, given the nature of soft tissue grafts.

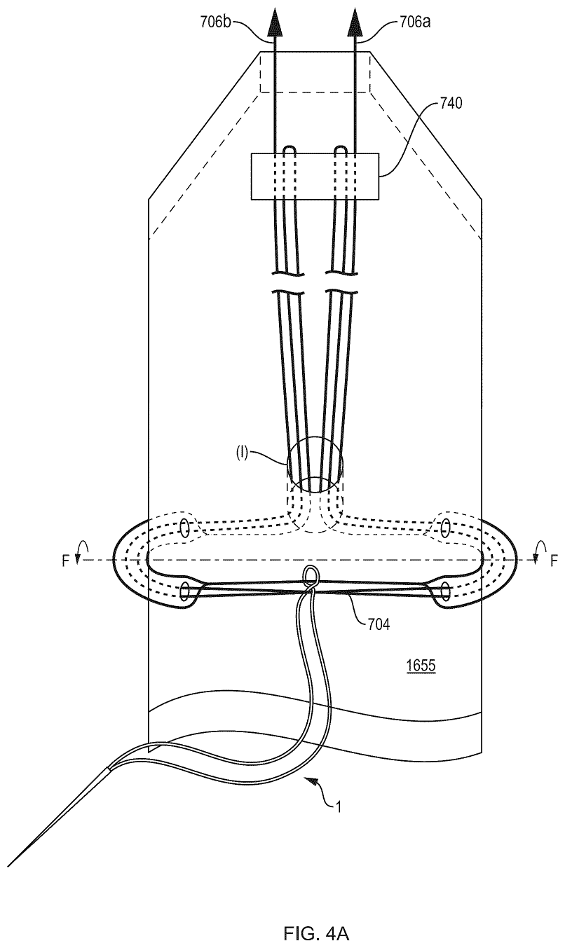

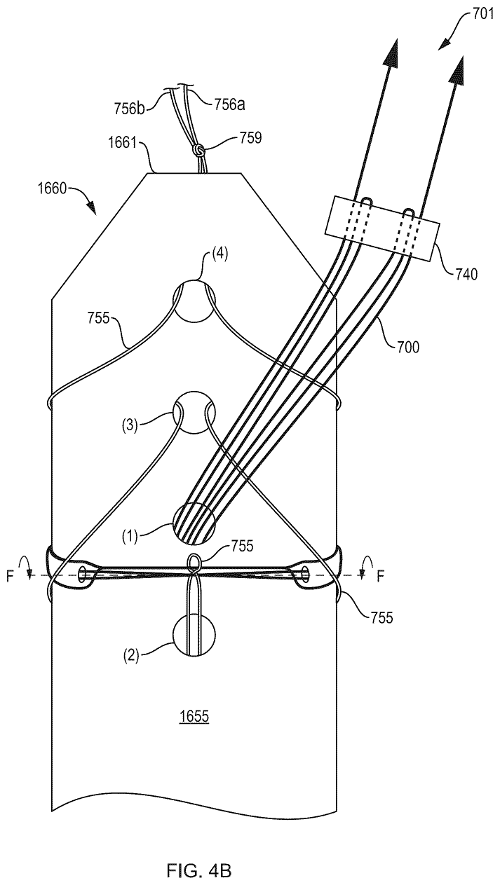

The adjustable loop construct 700 may then form a whipstitch, shown in FIG. 3E and FIG. 4A. The adjustable loop construct saddle end 704 may be spread or separated to wrap around both external side surfaces of graft 1650 and then flip or loop over the graft free end 1660, the button anchor 740 and tool 1725 to the top surface 1655. This may place the saddle end 704 between the clamped end 1670 and the first location (1), best seen in FIG. 3D and FIG. 4A.

Turning now to FIG. 3F, threading member 760 may be passed again (second pass) from the graft top surface 1655 to the bottom surface 1675, at a second location (2), directly adjacent to saddle end 704. This locks the location of saddle end 704 in place along the graft 1650, preventing the saddle end 704 from sliding along the graft 1650. Second location (2) may be approximately coincident with saddle end 704, and between the clamped end 1670 and first location (1). The adjustable loop construct 700 in now fixedly attached to the graft 1650. The second pass may be orthogonal to the graft longitudinal axis (Y-Y) and may define the furthest-most pass from the free end 1660. Consider now, that without any additional steps in the method, pulling on the suspension construct end 701 to draw the graft 1650 through a bone tunnel would apply tension adjacent the first and second locations (1) and (2). Should free end 1660 catch on the sides of the bone tunnel, the graft may fold (F) as illustrated in FIG. 4A.

The flexible passing loop 755 may now form a running whipstitch along graft 1650, progressively moving towards the free end 1660, the running whipstitch preferably including at least two whipstitch passes (a third and a fourth pass of the attachment method) through the graft 1650. This running whipstitch forms a plurality of axially spaced circumferential wraps around the graft free end 1660. Tensioning this plurality of axially spaced circumferential wraps forms the graft free end 1660 into a more cylindrical shape, for easier passing through the prepared bone tunnel. Tension on these whipstitches may further taper the graft free end 1660, for easier passing through the prepared bone tunnel. This running whipstitch may include at least two passes and acts to mitigate attachment rip-stopping (adjustable loop construct 700 and flexible loop 755 from cheese-wiring out of graft). Passing loop whipstitches run progressively towards the free end 1660 up to the free end terminal edge 1661. Loop 755 may then be tied in a knot 759 and cinched tightly to taper tapered free end 1660. The threading member 760 may then be removed.

The next steps in the method are best viewed in a top view, and so the figures shift accordingly. For reference, FIG. 4A illustrates a top-down view in an arrangement similar to that shown in FIG. 3E. FIG. 4B illustrates a top-down view of in a similar arrangement after that shown in FIG. 3J, with the passing loop 755 trimmed and knot 759 tied, resulting in two flexible strand ends 756a, 756b. Graft 1650 coupled as shown in FIG. 4B may then be drawn into and along a bone tunnel (not shown), via drawing on tool 725 or ends 706a, 706b. Consider however, that without any additional steps in the method, pulling on the suspension construct end 701 (via tool 725) to draw the graft 1650 through a bone tunnel may still apply tension adjacent the first and second locations (1) and (2). This may cause the graft 1650 to get stuck, may damage the graft 1650 and may hinder loop reduction and the overall repair. The inventors have found that pulling on both ends 701 and ends 756a and or 756b together, helps to prevent this folding. However, pulling on both ends in unison, arthroscopically, along ports and bone tunnels is not as easy as it sounds. Therefore, the inventors have found that coupling specific strands of the two ends (710, 756) together may be preferable. Therefore, the method may include additional steps to operatively couple the two ends so that they may remain coupled without frustrating the rest of the tissue repair.

A first example method of additional steps to limit the graft 1660 from folding so as to apply tension concomitantly on the adjustable construct 700 and to ends of the flexible loop ends 756a, 756b is now disclosed. This method moves the tensioning location towards the free end edge 1661 of graft. This may be achieved by coupling at least one of the passing loop ends 756a, 756a to the adjustable loop construct 700. While drawing the graft 1650 through the bone tunnel using the system 100, tension is now applied to both the adjustable construct 700 and flexible loop end (756a, 756b) simultaneously. Coupling may be achieved by various means including stitching them together using a needle or suture passer. At least one of ends 756a, 756b may be threaded through a portion or portions of strand 130 (707a, 708a, 709a, 707b, 708b, 709b). In some embodiments coupling means may be provided such as clips or staples, and the method may include clipping or stapling a portion of strand 130 to at least one end 756a, 756b.

It may be preferable to couple at least one of the ends 756a, 756b to a static strand of the adjustable construct, for example 709a or 709b of adjustable loop construct 700. If coupled to a static strand, the end or ends 756a, 756b may preferably not frustrate the adjustable construct 700 during loop reduction. During loop reduction, non-static strands need to readily slide relative to locking passages and/or graft, and should ends 756a, 756b be threaded through non-static strands, this sliding may be frustrated. Therefore, a first step in the first example method may include marking all the strands (706a, 706b, 707a, 707b, 709a, 709b) and then slightly reducing adjustable construct 700 to determine which strand is static. In some example methods, the static strand may be provided pre-marked.

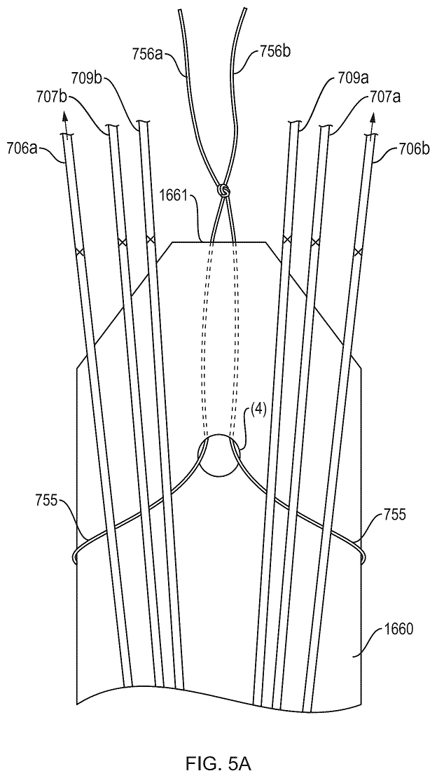

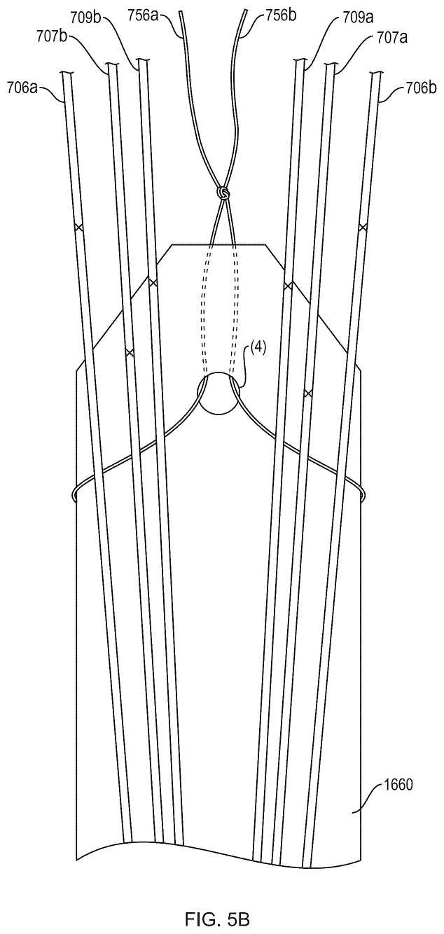

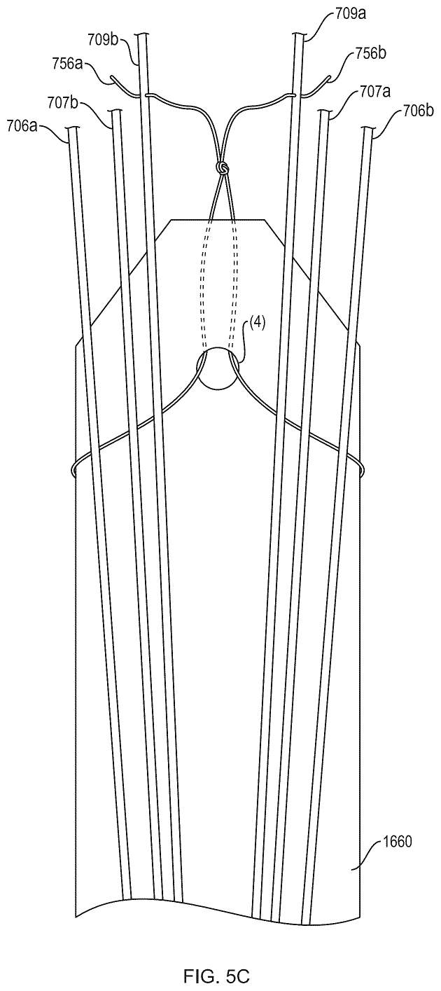

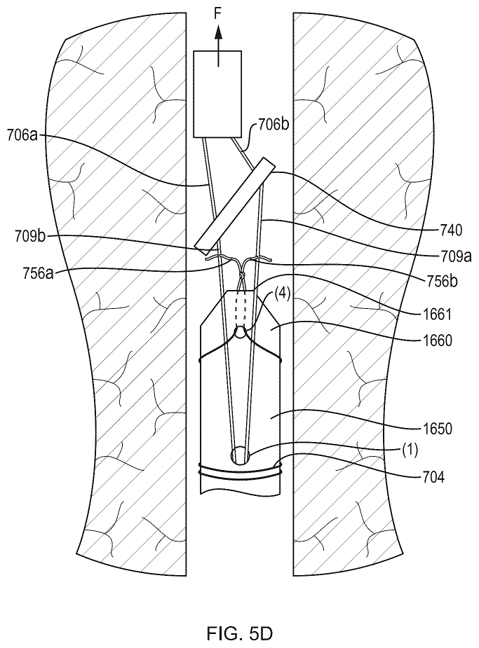

Example marks “X” are shown in FIG. 5A. Tension on ends 706a, 706b (see arrows) may reduce the adjustable loop construct 700 a small amount sufficient to see which marks “X” move (non-static strands) and which do (static strands), as illustrated in FIG. 5B. Static strands 709a, 709b are static strands and therefore do not move relative to the graft 1660. This principal is translatable to other adjustable loop construct, which may have more or less static and non-static strands.

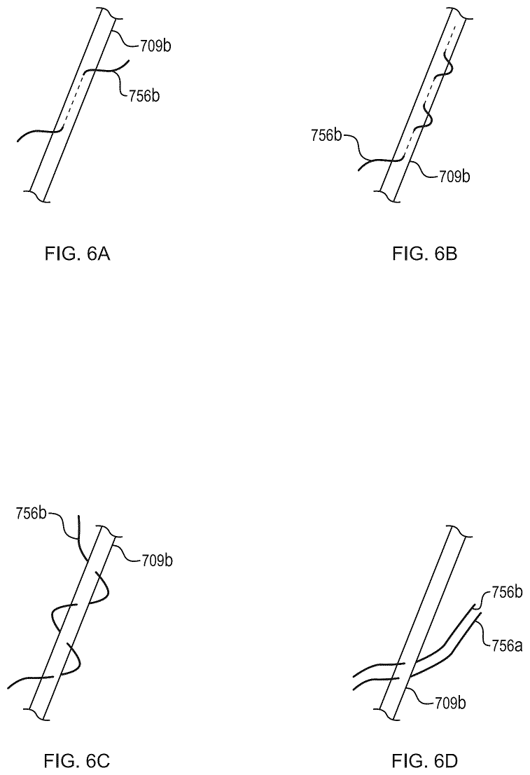

Upon determining at least one static strand, at least one passing loop end (756a, 756b) may then be coupled to this static strand (709a, 709b). Consider however that while the static strand does not move relative to the graft 1650, it may slide closer to the anchor 740. Therefore, it may be preferable to couple the at least one end 756a, 756b to the static strand (709a, 709b) directly adjacent to graft free end 1660, so that the anchor 740 does not run into the coupled ends during reduction. FIG. 5C shows each end 756a, 756b having been threaded through a corresponding static strand (709a, 709b). In some embodiments the at least one end (756a, 756b) may be spliced through and along the static strand (FIG. 6A). Adjustable loop construct 700 may be provided with a flexible snare loop that both indicates at least one of the static strands (709a, 709b) and then captures and draws at least one flexible loop end (756a, 756b) into and long a hollow core of static strands. In some embodiments the at least one end (756a, 756b) may be repeated spliced through and along the static strand (FIG. 6B). In some embodiments the at least one end (756a, 756b) may be repeatedly passed though the static strand (FIG. 6C). Optionally, both flexible loop ends 756a, 756b may be coupled to the same static strand in any manner disclosed herein (FIG. 6D).

FIG. 5D illustrates, in simplified form, the graft 1660 being drawn through a bone tunnel. Passing construct ends 756a and 756b are both shown coupled to static strands 709a, 709b such that upon drawing the system 100 and graft 1650 through the bone tunnel, there in concomitant tension on a portion of the graft adjacent the first location (1) where the saddle end 704 extends around graft 1660 and also where the passing construct ends extend from graft 1660 (location (4)), closer to the free end 1660. By pulling on tool 725 (F) this concomitant tension helps to prevent the graft 1650 from folding.

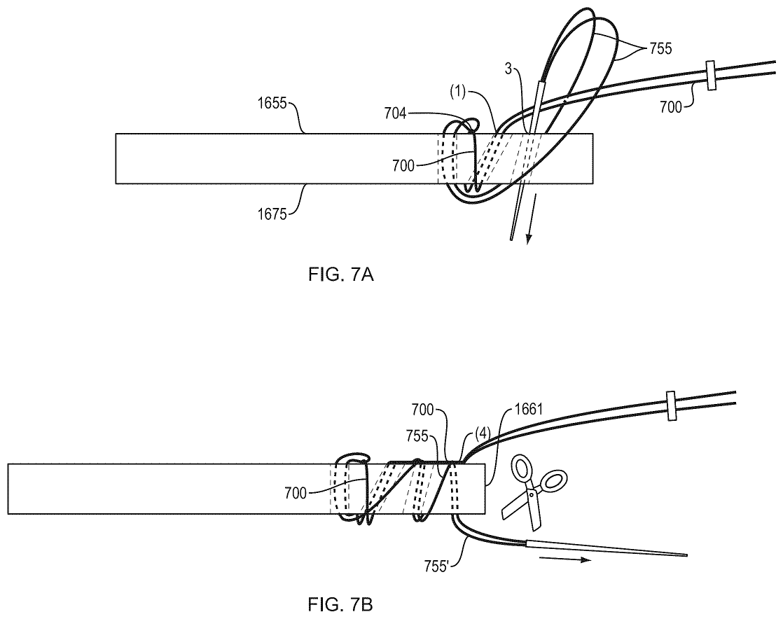

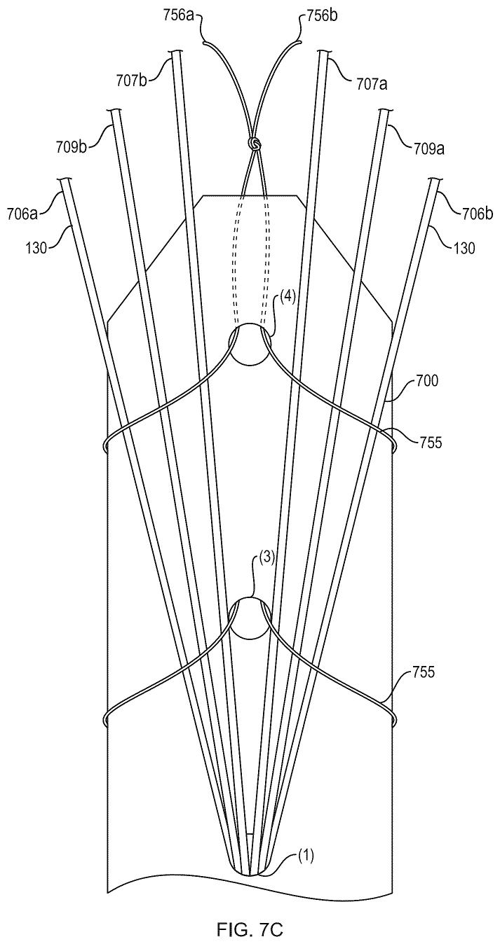

Another example method for prevent the graft from folding is illustrated in FIG. 7A-7C. This method may be combined with the method shown in FIG. 5A-5C or may stand alone. This method follows the method disclosed in FIGS. 3A-3H, except that when forming the running whipstitches with the passing loop 755, the passing loop 755 extends over the adjustable loop strands 130 (707a, 707b, 708a, 708b, 709a, 709b) to as to hold these strands 130 against the surface of graft 1660.

The running whipstitch preferably include at least two whipstitch passes (a third and a fourth pass of the attachment method) through the graft 1650. This running whipstitch forms a plurality of axially spaced circumferential wraps around both the graft free end 1660 and strands of the adjustable loop construct 700. This running whipstitch may include at least two passes and acts to mitigate attachment rip-stopping (adjustable loop construct 700 and flexible loop 755 from cheese-wiring out of graft). Whipstitches run progressively towards the free end 1660. Whipstitches may be formed by looping the flexible loop 755 around from the bottom surface 1675, around end 1660 and passing the adjustable loop construct anchor 750 through loop 755 before placing the threading member 760 on the top surface 1655, as shown in FIG. 7A. Threading member 760 may then pass from the top surface 1655 to the bottom surface 1675 again. This may be at a location (3) between free end 1660 and location (1). This may circumferentially wrap the passing loop 755 around the graft 1650, over and across the adjustable loop 700 that is wrapped around the graft external side surfaces and the saddle end 704 and also over the length of adjustable loop construct that extends between the saddle end 704 and anchor 740.

This step may be repeated to form at least one other pass of the running whipstitch. With the running whipstitch extending over the adjustable loop construct 700, as shown in FIG. 6B. This may be repeated until the whipstitch passes are close to the terminal edge 1661 of the free end 1660. Loop 755 may then be tied in a knot and cinched tightly to further taper tapered free end 1660.

FIG. 7C illustrated a top-side view of a graft free end 1660 of FIG. 7B, showing passing loop 755 extending over strands 130 for at least 2 whipstitches. In this example method, strands 130 of adjustable loop construct 700 may be split or separated either side of the midline of graft 1650 and either side of the passes (3) and (4). This may balance tension on the graft 1650 during adjustable loop reduction. In other embodiments, all the strands 130 may be on one side of graft 1650.

Turning now to a more specific example, a method of attaching may include forming or obtaining adjustable loop construct 700 assembled with a cortical button 750 at one end and coupled to a passing construct 750 at saddle end 704. Threading member 760 may pierce the graft top surface 1655 and draw the passing loop 755 of passing construct 750 from the graft top surface 1655 through the thickness of graft 1650 to the lower external surface 1675, at a first location (1). First location (1) may be about 1.5 cm-2 cm from terminal edge 1661 of free end 1660. An adjustable loop construct whipstitch may now be formed (FIG. 3E). Threading member may be passed again (second pass) from the graft top surface 1655 to the bottom surface 1675, at a second location (2), directly adjacent to saddle end 704 to locks the location of saddle end 704 along the graft 1650 and prevents the saddle end 704 from sliding along the graft 1650. Second location (2) may be approximately coincident with saddle end 704, and between the clamped end 1670 and first insertion location (1).

The passing loop 755 may now form a running whipstitch that progressively stitches towards the free end 1660, the running whipstitch preferably including at least two whipstitch passes (a third and a fourth pass of the attachment method) through the graft 1650. This running whipstitch forms a plurality of axially spaced circumferential wraps around the graft free end 1660. In some example methods, the adjustable loop construct 700 may first be placed on graft top surface 1655, and the running whipstitches passed over strands 130 of the adjustable loop construct 700, so that the running whipstitches hold these adjustable loop construct strands 130 against the graft. Tensioning this plurality of axially spaced circumferential wraps may hold the adjustable loop construct 700 against the graft free end top surface 1655 along the graft free end 1660 and closer to the free end edge 1661. These running whipstitch may include at least two passes and acts to mitigate attachment rip-stopping (adjustable loop construct 700 and flexible loop 755 from cheese-wiring out of graft). Thereafter the threading member 760 may be removed leaving two loop ends 756a, 756b.

Optionally ends 756a, 756b may be coupled to a portion of the adjustable loop construct strand 130. This may preferably be a static strand of the adjustable loop construct 700. The static strand may first be determined by marking all strands extending along the construct 700 and then slightly reducing the adjustable construct 700 to observe with strands move (non-static) and which do not. At least one of the ends 756a, 756b may not be coupled to at least one of the static strands. In some example methods, a first end 756a is coupled to a first static strand, while a second end 756b is coupled to a second static strand. In some other example embodiments, the static strands may be provided pre-marked, or have a snare tool assembled thereto. Coupling the at least one end 756a, 756b include stapling or clipping them. Coupling the at least one end may include threading the at least one end 756a, 756b through at least one static strand. Coupling the at least one end may include threading the at least one end 756a, 756b into and along a hollow core of the at least one static strand. Graft 1650 coupled to adjustable loop construct 700 and ends 756a, 756b may then be drawn through a bone tunnel that may be prepared ahead of time. Drawing on adjustable loop end only no draws graft 1650 while concomitantly applying tension to the graft free end 1660, so avoid the graft from folding.

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

1. A method of coupling a suspensory fixation system to a graft, the suspensory fixation construct including an adjustable loop construct and a passing loop construct linked thereto, the method comprising:
stitching the adjustable loop construct through and around the graft at a first location along the graft, using the passing construct;
stitching the passing construct through the graft adjacent the first location to secure the adjustable loop construct to the graft;
with the adjustable loop construct secured, laying a length of the adjustable loop along an outer surface of the graft and stitching the passing loop construct around and through the graft at a second location along the graft, the second location disposed between the first location and a free end of the graft and wherein stitching the passing loop construct at the second location wraps a strand of the passing loop construct around and over the length of the adjustable loop construct and holds the length of the adjustable loop against the graft adjacent the second location.

2. The method of claim 1, wherein the first location is spaced away from the graft free end.

3. The method of claim 1 further comprising stitching the passing loop construct around and through the graft at a third location along the graft, the third location disposed between the second location and the free end of the graft and wherein stitching the passing construct at the third location wraps the passing loop construct again around and over the length of the adjustable loop construct and holds the length of the adjustable loop against the graft adjacent the third location.

4. The method of claim 1 further comprising coupling an end of the passing loop construct to the adjustable loop construct after stitching the passing construct around and through the graft at the second location.

5. The method of claim 4 further comprising drawing the adjustable loop construct along a bone tunnel after coupling the passing construct to the adjustable loop construct, and thereby drawing the graft free end along the bone tunnel.

6. The method of claim 4 wherein the adjustable loop construct includes a static leg and wherein coupling the passing loop construct to the adjustable loop construct comprises coupling a flexible strand of the passing loop construct to the static leg.

7. The method of claim 6 wherein the static leg is marked.

8. The method of claim 6 further comprising reducing the adjustable loop construct to determine the static leg before coupling the flexible strand of the passing construct to the static leg.

9. The method of claim 6 wherein coupling the flexible strand of the passing construct to the static leg comprises stitching the flexible strand through the static leg.

10. The method of claim 1 further comprising drawing the adjustable loop construct along a bone tunnel, and thereby drawing the graft free end along the bone tunnel.

11. The method of claim 1 wherein the length of the adjustable loop construct includes multiple loop lengths of the adjustable loop construct and wherein stitching the passing construct at the second location inserts the passing construct through the graft at the second location and splits the multiple loop lengths so that they extend along both sides of the second location.

12. A method of coupling a graft suspension system to a graft comprising:
obtaining the graft and the graft suspension system, the graft suspension system including;
a tissue anchor having a plurality of openings therethrough;
an adjustable loop construct formed with a flexible strand, a first end of the adjustable loop construct coupled to the tissue anchor via the plurality of openings, the flexible strand including at least one locking passage; and
a passing construct including a flexible strand and needle, the flexible strand separately formed from the adjustable loop construct flexible strand and coupled to a second end of the adjustable loop construct defining a first link, at an opposing end to the adjustable loop construct first end;
coupling the adjustable loop construct to the graft at a first location along the graft;
coupling the passing construct to the graft at a second location along the graft; and
linking the passing construct flexible strand to the adjustable loop construct after coupling the adjustable loop construct to the graft at the first location and coupling the passing construct to the graft at the second location, such that the flexible strand and the adjustable loop construct are linked by the first link and a second link, at separate locations; and
drawing the graft suspension system and the graft through a bone tunnel.

13. The method of claim 12 wherein coupling the passing construct includes forming at least two whipstitches along the graft free end with the passing construct.

14. The method of claim 12 wherein linking the passing construct flexible strand to the adjustable loop construct comprises coupling the passing construct flexible strand to a static leg of the adjustable loop construct.

15. The method of claim 12 wherein linking the passing construct flexible strand to the adjustable loop construct comprises at least one of passing or splicing the passing construct flexible strand to the adjustable loop construct.

16. The method of claim 12 wherein linking includes passing a first end of the passing construct flexible strand through the adjustable loop construct flexible strand at a first location and also passing a second end of the passing construct flexible strand through the flexible strand of the adjustable loop construct at a second, different location.

17. The method of claim 12 wherein the adjustable loop construct includes a static leg, and wherein linking included passing an end of the passing construct flexible strand through the static leg.

18. The method of claim 17 further comprising adjusting the adjustable loop construct so as to identify the static leg before linking.

19. The method of claim 17 wherein the static leg includes a mark for identifying the static leg.

20. The method of claim 12 wherein while coupling the passing construct to the graft at a second location along the graft; passing the passing construct flexible strand over the adjustable loop construct.
